(19) United States
(12) Patent Application Publication
(10) Pub. No.: US 2026/0363648 A1
(43) Pub. Date: Nov. 19, 2026

(54) CHESTPIECE FOR STETHOSCOPE, ELECTRONIC STETHOSCOPE AND USE

(71) Applicant: LAPSI HEALTH B.V., Amsterdam (NL)

(72) Inventors: Diana VAN STIJN, Amsterdam (NL); Toni LEINONEN, Amsterdam (NL); Jhonatan BRINGAS DIMITRIADES, Amsterdam (NL)

(21) Appl. No.: 19/126,148

(22) PCT Filed: Oct. 30, 2023
(86) PCT No.: PCT/EP2023/080215

§ 371 Date: Apr. 30, 2025

(30) Foreign Application Priority Data
Oct. 31, 2022 (FI) 20225971

(51) Int. Cl. A61B 7/04; A61B 5/024; A61B 5/026; A61B 5/0295

(52) U.S. Cl. CPC A61B 7/04; A61B 5/02416; A61B 5/0261; A61B 5/0295

(57) ABSTRACT

A chestpiece for an electronic stethoscope, an electronic stethoscope and use of a photoplethysmography sensor in the chestpiece. The chestpiece comprises a body, an audio chamber inside the body for detecting auscultated sound from a patient body, a first microphone provided in connection with the sound chamber for converting the auscultated sound into an auscultated sound signal, and a processing unit configured to receive the auscultated sound signal from the first microphone. The chestpiece further comprises a photoplethysmography sensor arranged to detect blood flow volume in the patient body.

### FIELD OF THE INVENTION

The present invention relates to a chestpiece for an electronic stethoscope and more particularly to a chestpiece accord to preamble of claim 1. The present invention further relates to an electronic stethoscope, and more particularly to an electronic stethoscope according to preamble of claim 15.

### BACKGROUND OF THE INVENTION

Prior art electronic stethoscopes are directed to harvest information based on auscultated sounds of a patient body. The auscultated sounds are provided with a chestpiece of the electronic stethoscope. The chestpiece comprises a microphone configured to convert the auscultated sounds to audio signal. The prior art electronic stethoscopes comprise different kinds of amplifiers, filters and the like for processing the audio signal received from the microphone. Accordingly, the audio signal is further processed and analysed with a processing unit in order to provide specialized auscultated sound for a doctor.

One of the problems associated with the prior art is that the information of the patient provided by the prior art electronic stethoscopes is limited. Accordingly, the prior art electronic stethoscopes enable only limited analysis of physical condition of the patient.

### BRIEF DESCRIPTION OF THE INVENTION

An object of the present invention is to provide a chestpiece, an electronic stethoscope and a use of a photoplethysmography sensor so as to solve or at least alleviate the prior art disadvantages.

The objects of the invention are achieved by a chestpiece which is characterized by what is stated in the independent claim 1. The objects of the invention are further achieved by an electronic stethoscope which is characterized by what is stated in the independent claim 15.

The preferred embodiments of the invention are disclosed in the dependent claims.

The invention is based on the idea of providing a chestpiece for an electronic stethoscope. The chestpiece comprises a body, an audio chamber inside the body for detecting auscultated sound from a patient body, a first microphone provided in connection with the sound chamber for converting the auscultated sound into an auscultated sound signal, and a processing unit configured to receive the auscultated sound signal from the first microphone. According to the present invention the chestpiece further comprises a photoplethysmography sensor arranged to detect blood flow volume in the patient body. The body of the chestpiece comprises an outer front surface arranged to be placed against the patient body. The audio chamber is provided in connection with the outer front surface for detecting the auscultated sound from the patient body via the outer fron surface. The outer front surface comprises a diaphragm arranged to form front chamber wall of the audio chamber. The photoplethysmography sensor arranged in connection with the diaphragm.

The chestpiece of the present invention enables both detecting auscultated sounds from the patient body and at the same time detecting blood flow volume in the patient body. Especially, the chestpiece enables detecting auscultated sounds and blood flow volume in the patient body at a specific location simultaneously.

In some embodiments, the photoplethysmography sensor comprises an optical emitter configured to emit light and a light detector arranged to detect light. In some embodiments, the photoplethysmography sensor comprises a pulse oximeter sensor.

The blood flow volume or changes in the blood flow volume are detected by emitting light to skin of the patient or illuminating the skin of the patient with the optical emitter. Then light reflected from the skin of the patient is detected or measured with the light detector. The light detector thus measures intensity non-absorbed light reflected from the tissue of the patient. Thus, blood flow volume or change of blood flow volume is detected with the photoplethysmography sensor.

The photoplethysmography sensor comprises one or more optical emitters and a light detector.

In some embodiments, the optical emitter is configured to emit infrared light. The optical emitter is an infrared optical emitter.

In some embodiments, the optical emitter comprises an infrared light emitting diode.

Infrared light provides effective penetration depth into the skin of the patient.

In some embodiments, the optical emitter is configured to emit green light or infrared green light.

The optical emitter comprises green light emitting diode configured to emit green light or green infrared light.

Green light is less susceptible to motion disturbances than infrared light.

In some embodiments, the optical emitter is configured to emit red light, infrared red light, yellow light or infrared yellow light.

The optical emitter comprises red light emitting diode configured to emit red light or red infrared light. Alternatively, the optical emitter comprises yellow light emitting diode configured to emit yellow light or yellow infrared light.

The light detector comprises a photodiode. The photodiode measures intensity of emitted light reflected from the patient body.

In some embodiment, the chestpiece comprises two or more photoplethysmography sensors.

Two or more photoplethysmography sensors enable calculating combined results of blood flow volume. Alternatively, the two or more photoplethysmography sensors enable selecting a photoplethysmography signal having highest quality, or eliminating or deleting erroneous photoplethysmography signals. Accordingly, reliability is increased.

In some embodiments, the body comprises an outer front surface arranged to be placed against the patient body. The audio chamber is provided in connection with the outer surface for detecting the auscultated sound from the patient body via the outer front surface. The photoplethysmography sensor is arranged in connection with the outer front surface of the body.

Accordingly, the outer front surface of the chestpiece is arranged to be placed against the patient body or skin and the auscultated sounds and the blood flow volume are detected simultaneously and at the same location of the patient body.

In some embodiments, the photoplethysmography sensor is arranged on the outer front surface of the body.

The photoplethysmography sensor on the outer front surface of the chestpiece is placed directly against the patient body or skin when auscultated sounds of the patient body are detected by utilizing the audio chamber.

In some embodiments, the outer front surface comprises a diaphragm arranged to form a front chamber wall of the audio chamber.

The diaphragm is arranged to form at least part of the outer front surface of the body of the chestpiece. The diaphragm is further arranged to close the audio chamber at the outer front surface of the chestpiece. Accordingly, the diaphragm is arranged between the chamber space of the audio chamber and outside of the chestpiece.

The diaphragm is provided as a flexible diaphragm. Alternatively, the diaphragm is made of flexible material. Flexibility of the diaphragm enables the diaphragm to move or vibrate as a response to body sounds of the patient when the diaphragm is placed against the patient body. The movement or vibration of the diaphragm generates acoustic pressure waves into the audio chamber which are further detected with the first microphone.

The diaphragm is arranged to form part of the outer front surface and the body of the chestpiece is arranged to form a part of the outer front surface. The photoplethysmography sensor is arranged to part of the outer front surface formed by the body.

The photoplethysmography sensor is preferably arranged on the same level or flush with the diaphragm on the outer front surface such that the diaphragm and the photoplethysmography sensor are both against the patient body or skin simultaneously when the outer front surface is place against the patient skin.

Alternatively, photoplethysmography sensor is arranged protrude outwards from the level of the diaphragm on the outer front surface such that it is ensured that the photoplethysmography sensor is against the patient skin when the outer front surface and the diaphragm are place against the patient skin.

The photoplethysmography sensor, or the optical emitter and light detector thereof, comprises a sensor front surface which is arranged to be placed against the patient skin. The sensor front surface is arranged flush with the diaphragm or outwards from the level of the diaphragm.

In some embodiments, the photoplethysmography sensor is arranged in connection with the diaphragm.

In some other embodiments, the photoplethysmography sensor is arranged to the diaphragm.

Accordingly, the diaphragm provided to the outer front surface of the chestpiece is utilized for the photoplethysmography sensor. Thus, no separate elements are provided on the outer front surface.

In some embodiments, the photoplethysmography sensor is attached to the diaphragm.

Thus, the photoplethysmography sensor is arranged to move together with the diaphragm such that is remains against the patient skin.

In some embodiments, the photoplethysmography sensor is arranged to pierce the diaphragm.

In some embodiments, the diaphragm comprises an opening, and the photoplethysmography sensor is arranged to the opening.

The diaphragm comprises an outer diaphragm surface arranged to form at least part of the outer front surface of the body, and an inner diaphragm surface towards the audio chamber.

In some embodiments, the photoplethysmography sensor is arranged on the side of the inner diaphragm surface of the diaphragm and arranged to detect blood flow volume in the patient body through the diaphragm. The photoplethysmography sensor is directed towards the diaphragm such that the light from the optical emitter is emitted through the diaphragm and detected with the light detector through the diaphragm.

The photoplethysmography sensor is arranged on the inner diaphragm surface. Alternatively, the photoplethysmography sensor is arranged inside the audio chamber or to an audio chamber wall spaced apart from the diaphragm. The photoplethysmography sensor is directed towards the diaphragm such that the light from the optical emitter is emitted through the diaphragm and detected with the light detector through the diaphragm.

Accordingly, the photoplethysmography sensor may be arranged inside the body of the chestpiece.

In an alternative embodiment, the optical emitter is arranged on the side of the inner diaphragm surface of the diaphragm and arranged to emit light through the diaphragm. The light detector is arranged on the side of the outer diaphragm surface of the diaphragm or on the outer diaphragm surface of the of the diaphragm.

Thus, the optical emitter is arranged inside the body of the chestpiece. In a further alternative embodiment, the light detector is arranged on the side of the inner diaphragm surface of the diaphragm and arranged to detect light through the diaphragm, and the optical emitter is arranged on the side of the outer diaphragm surface of the diaphragm or on the outer diaphragm surface of the diaphragm.

Thus, the light detector is arranged inside the body of the chestpiece.

In the above embodiments, the diaphragm is provided light permeable, transparent or translucent. Thus, light emitted by the optical emitter can travel through diaphragm. Similarly, the light detector can detect or measure reflected flight through the diaphragm.

The diaphragm may also be provided with an optical element which is configured to be light permeable, transparent or translucent. Thus, light emitted by the optical emitter can travel through diaphragm via the optical element. Similarly, the light detector can detect or measure reflected flight through the diaphragm via the optical element.

The optical element is provided as part of the diaphragm or arranged to form an area of the diaphragm. Therefore, the functioning of the diaphragm is not affected.

In some embodiments, the diaphragm comprises an optical element. The optical element being a light permeable element, the photoplethysmography sensor is arranged on the side of the inner diaphragm surface of the diaphragm and configured to emit through the optical element and detect light through the optical element.

In some other embodiments, the diaphragm comprises a first optical element. The first optical element being a light permeable element, the optical emitter is arranged on the side of the inner diaphragm surface of the diaphragm and arranged to emit light through the first optical element.

In some further embodiments, the diaphragm comprises a second optical element. The second optical element being a light permeable element, the light detector is arranged on the side of the inner diaphragm surface of the diaphragm and arranged to detect light through the second optical element.

In some yet further embodiments, the diaphragm comprises a first optical element. The first optical element being a light permeable element, the optical emitter is arranged on the side of the inner diaphragm surface of the diaphragm and arranged to emit light through the first optical element. The diaphragm also comprises a second optical element. The second optical element being a light permeable element, the light detector is arranged on the side of the inner diaphragm surface of the diaphragm and arranged to detect light through the second optical element.

In some embodiments, the optical element is a lens.

In some embodiments, the first optical element is a first lens.

In some embodiments, the second optical element is a second lens.

In some embodiments, the first and second lens have different optical characteristics.

In some embodiments, the first and second lens have refractive index and/or different focal lengths or focal points.

In some alternative embodiments, the optical element may be omitted. The diaphragm is provided as light permeable or at least partly light permeable. Thus, the photoplethysmography sensor or the light detector and/or the light emitter may be arranged as disclosed above.

In the above embodiments, the diaphragm may be provided as the optical element.

In some embodiments, the diaphragm comprises an outer diaphragm surface arranged to form at least part of the outer front surface of the body, and an inner diaphragm surface towards the audio chamber and the photoplethysmography sensor, or the light detector and the light emitter, is arranged on the outer diaphragm surface of the diaphragm.

In some embodiments, the photoplethysmography sensor is configured to generate a photoplethysmography signal based on the detected blood flow volume in the patient body, and the processing unit is configured to receive the photoplethysmography signal from the photoplethysmography sensor.

The processing may further process the photoplethysmography signal. In some embodiments, the chestpiece is configured to simultaneously generate the photoplethysmography signal with the photoplethysmography sensor and the auscultated sound signal with the first microphone.

Generating the photoplethysmography signal simultaneously with the auscultated sound signal enables modulating the photoplethysmography signal with the auscultated sound signal, or the auscultated sound signal with the photoplethysmography signal, of a same time window. Further, the photoplethysmography signal and the auscultated sound signal may be compared or processed to find correlations for analysing health factors of the patient.

In some embodiments, the chestpiece comprises a wireless communication module configured to transmit the photoplethysmography signal and the auscultated sound signal from the chestpiece to an external receiver.

In some other embodiments, the processing unit is configured to process the photoplethysmography signal and the auscultated sound signal into output data and the wireless communication module is configured to transmit the output data to an external receiver.

Accordingly, the chestpiece is provided as a separate wireless device enabling remote analysis and also convenient use without sound tubes.

Processing the auscultated sound signal comprises at least one of filtering and amplification of the auscultated sound signal.

The output data comprises photoplethysmography data generated from the photoplethysmography signal in the processing unit, and auscultated data generated from the auscultated sound signal in the processing unit.

In some embodiments, the wireless communication module comprises a short-range wireless communication module, or a Bluetooth communication module, or a Wi-Fi communication module.

The wireless communication module enables providing a data transfer connection between the chestpiece and user device, such as mobile user device, computer, server or headphones.

It should be noted that the wireless communication module may be any known or future wireless communication module utilizing any wireless communication protocol for data transfer without departing the present invention.

The present invention further relates to an electronic stethoscope. The electronic stethoscope comprises a chestpiece. The chestpiece comprises a body, an audio chamber for detecting auscultated sound from a patient body, a first microphone provided in connection with the sound chamber for converting the auscultated sound into an auscultated sound signal, a photoplethysmography sensor arranged to detect blood flow volume in the patient body and generate a photoplethysmography signal based on the detected blood flow volume, a processing unit configured to receive the auscultated sound signal from the first microphone and the photoplethysmography signal from the photoplethysmography sensor, and a wireless communication module configured to transmit in wireless manner from the chestpiece the photoplethysmography signal and the auscultated sound signal or output data generated in the processing unit from the photoplethysmography signal and the auscultated sound signal. The body of the chestpiece comprises an outer front surface arranged to be placed against the patient body. The audio chamber is provided in connection with outer front surface for detecting the auscultated sound from the patient body via the outer front surface. The outer front surface comprises a diaphragm arranged to form a front chamber wall of the audio chamber. The photoplethysmography sensor is arranged in connection with the diaphragm. The electronic stethoscope further comprises an external user device configured to receive the photoplethysmography signal and the auscultated sound signal or the output data from the chestpiece in wireless manner.

The electronic stethoscope enables both detecting auscultated sounds from the patient body and at the same time detecting blood flow volume in the patient body and transferring the detected auscultated sound information and the blood flow volume information in wireless manner to the external user device.

In some embodiments, the external user device is a mobile user device or a computer configured to receive the photoplethysmography signal and the auscultated sound signal or the output data from the chestpiece in wireless manner.

The mobile user device or the computer is configured to display the detected photoplethysmography signal or photoplethysmography data derived from the photoplethysmography signal with a display device of the mobile user device or the computer. The mobile user device or the computer is configured to emit the auscultated sounds of the auscultated signal or auscultated sound data derived from the auscultated signal with a speaker device of the mobile user device or the computer.

Alternatively, the external user device is an audio output device to receive the photoplethysmography signal and the auscultated sound signal or the output data from the chestpiece in wireless manner.

The audio output device may be speaker device, headphones of the like. The audio output device is configured to emit the auscultated sounds of the auscultated signal or auscultated sound data derived from the auscultated signal. The audio output device comprises one or mor speakers.

In some embodiments, the external user device is the mobile user device or the computer configured to receive the photoplethysmography signal and the auscultated sound signal or the output data from the chestpiece in wireless manner. The mobile user device or the computer is further configured to transmit the photoplethysmography signal and the auscultated sound signal or the output data to the audio output device.

The mobile user device or the computer may be configured to transmit the photoplethysmography signal and the auscultated sound signal or the output data to the audio output device in wireless manner by utilizing a wireless communication protocol. The wireless communication protocol may be a short-range wireless communication protocol.

The mobile user device is configured to display the detected photoplethysmography signal or photoplethysmography data derived from the photoplethysmography signal with a display device of the user device.

The audio output device may be speaker device, headphones of the like. The audio output device is configured to emit the auscultated sounds of the auscultated signal or auscultated sound data derived from the auscultated signal. The audio output device comprises one or mor speakers.

In some embodiments, the mobile user device or the computer is configured to display the detected photoplethysmography signal or photoplethysmography data derived from the photoplethysmography signal with the display device of the mobile user device or the computer simultaneously as the speaker device of the mobile user device or the computer or the separate audio output device is configured to emit the auscultated sounds of the auscultated sounds of the auscultated signal or auscultated sound data derived from the auscultated signal.

In some embodiments of the electronic stethoscope, the chestpiece is a chestpiece as defined above.

The present invention further relates to use of a photoplethysmography sensor in a chestpiece of a wireless electronic stethoscope comprising a first microphone for detecting blood flow volume in a patient body simultaneously with detecting auscultated sound from the patient body.

An advantage of the invention is that it enables detecting auscultated sounds from the patient body and at the same time detecting blood flow volume in the patient body and transferring the detected auscultated sound information and the blood flow volume information in wireless manner to the external user device. The present invention further enables detecting simultaneously auscultated sounds and blood flow volume from the patient body and at the same location of the body by utilizing the chestpiece.

### BRIEF DESCRIPTION OF THE DRAWINGS

The invention is described in detail by means of specific embodiments with reference to the enclosed drawings, in which

FIG. 1 shows a schematic outer view of a chestpiece of an electronic stethoscope;

FIG. 2 shows a schematic cross-sectional view of the chestpiece of FIG. 1;

FIG. 3 shows a schematic front view of the chestpiece FIG. 1;

FIG. 4 shows a schematic cross-sectional view of the chestpiece of FIG. 1;

FIGS. 5 to 7 show schematically one embodiment according to the present invention;

FIGS. 8 to 10 show schematically another embodiment according to the present invention;

FIGS. 11 to 16 show schematically yet another embodiment according to the present invention;

FIGS. 17 to 26 show schematically further alternative embodiments according to the present invention.

### DETAILED DESCRIPTION OF THE INVENTION

FIG. 1 shows schematically one embodiment of a chestpiece 10 for an electronic stethoscope. The chestpiece 10 is configured to provide auscultated sounds of a patient body, for example heart, lungs, bowel, arteries and veins.

The chestpiece 10 comprises a body 12. The body comprises an outer top surface 16, an outer front surface 14 and outer side surface(s) 18 extending between the outer top surface 16 and the outer front surface 14. The outer front surface 14 is configured to be placed against a patient body or skin during health examination.

The outer front surface 14 is provided with a diaphragm 15 forming at least part of the outer front surface 14 of the body 12.

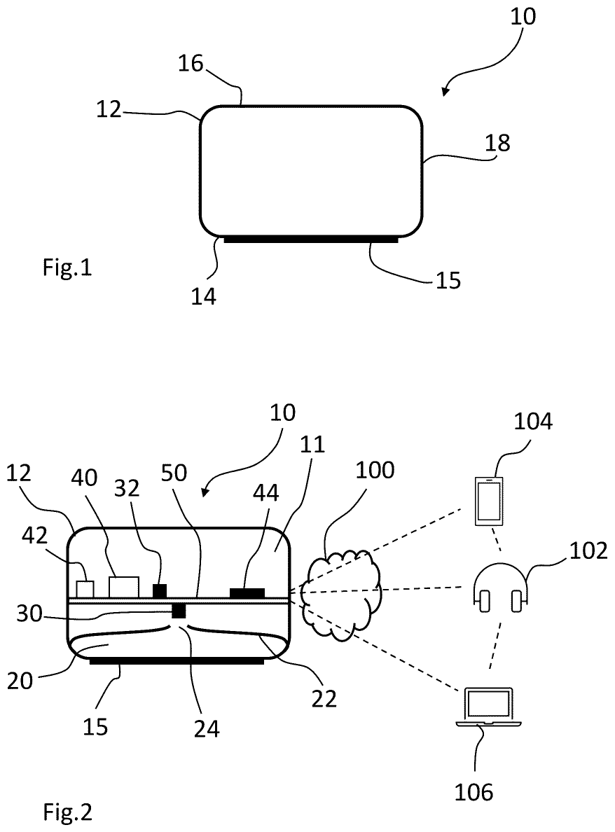

FIG. 2 shows schematically a cross-sectional view of the chestpiece 10 and a schematic configuration of an electronic stethoscope utilizing the chestpiece 10.

The chestpiece 10 comprises an audio chamber 20 inside the body 12. The audio chamber 20 is defined by audio chamber walls 22 and the diaphragm 15. The diaphragm 15 is arranged to separate the audio chamber 20 form the outside of the chestpiece 10 at the outer front surface 14. Thus, the audio chamber 20 is provided in connection with the outer front surface 14.

The diaphragm 15 has a planar shape.

The diaphragm 15 extends parallel to the outer front surface 14.

When the outer front surface 14 is placed against patient skin, the diaphragm 15 is also placed against the patient skin. The diaphragm 15 is provided as a flexible diaphragm. Flexibility of the diaphragm 15 enables the diaphragm 15 to move or vibrate as a response to body sounds of the patient when the outer front surface 14 and the diaphragm 15 is placed against the patient skin. The movement or vibration of the diaphragm 15 generates acoustic pressure waves into the audio chamber 20.

The audio chamber 20 has bell-shaped form, as shown schematically in FIG. 2. The diaphragm 15 or the outer front surface 14 is arranged to provide a bottom of the bell-shaped form. The end 24 of the bell-shaped audio chamber 20 is provided opposite the diaphragm 15. The bell-shaped form of the audio chamber 20 provides mechanical amplification of sound, sound waves or pressure waves generated with the diaphragm 15.

It should be noted that that the audio chamber 20 may also have a different shape. The audio chamber 20 may for example have a tapering or converging shape towards the top end 24 of the audio chamber 20. The top end 24 is arranged opposite the diaphragm 15.

The shape of the audio chamber 20 is arranged to provide amplification of the auscultated sounds towards the top end 24 of the audio chamber 20.

The chestpiece 10 comprises a first microphone 30 arranged in connection with the audio chamber 20. The first microphone is configured to capture auscultated sounds or the pressure waves provided by the diaphragm 15 and the audio chamber 20, and further convert the auscultated sounds into an auscultated sound signal.

The auscultated sound signal generated by the first microphone 30 is an electronic auscultated sound signal.

The first microphone 30 is arranged to in connection with the audio chamber 20 such that the first microphone 30 is arranged to capture the auscultated sounds from the audio chamber 20.

The first microphone 30 is arranged inside the audio chamber 20. Alternatively, the first microphone 30 is arranged to the audio chamber walls 22.

In the embodiment of FIG. 2, the first microphone 30 is arranged in connection with the top end 24 of the audio chamber 20.

The first microphone 30 is arranged to the top end 24 of the audio chamber 20.

In the embodiment of FIG. 2, the top end 24 of the audio chamber 20 comprises sound opening and the first microphone 30 is arranged to or in connection with the sound opening of the top end 24.

In some other embodiments the first microphone 30 is arranged to form the top end 24 of the audio chamber 20.

The first microphone 30 may be any kind of microphone device.

The chestpiece 10 comprises also a second microphone 32. The second microphone 32 is provided as an ambient microphone. The second microphone 32 is arranged capture or detect ambient sounds from the surroundings of the chestpiece 10. The second microphone 32 is arranged to generate an ambient sound signal or convert the ambient sounds into the ambient sound signal.

The second microphone 32 and the ambient sound signal is utilized for filtering the ambient sounds from the auscultated sound signal. Thus, ambient sounds or interference sounds do not disturb the diagnosis carried out based on the auscultated sounds.

It should be noted that the second microphone 32 may also be omitted. The chestpiece 10 further comprises a processing unit 40.

The processing unit 40 comprises one or more processors, and one or more memory elements. A software program comprising instructions for operating the chestpiece and the components thereof is stored to the one or more memory elements.

The processing unit may comprise a read only memory (ROM) and/or a random access memory (RAM). A basic input output system (BIOS) is stored in the ROM. The RAM is used as a work area of the processing unit or as a temporary storage apparatus for loading a software program for operating the chestpiece 10 and the components thereof.

It should be noted that the above is only one embodiment and the processing unit may be implemented in various ways without departing the present invention.

The processing unit 40 or software program stored therein may comprise instructions for processing the auscultated sound signal into auscultated sound data. The processing may comprise for example filtering and/or amplifying the auscultated sound signal and generating the auscultated sound data.

The processing unit 40 or software program stored therein is further configured to comprise instructions for operating the first microphone 30.

The processing unit 40 or software program stored therein is further configured to comprise instructions for operating the second microphone 32.

The chestpiece 10 further comprises a power unit 44. The power unit 40 comprises a battery. The battery is preferably a rechargeable battery.

The battery unit 44 further comprises a charging module arranged to charge the battery. The charging module is preferably provided as a wireless charging module arranged to charge the battery in wireless manner. The wireless charging module comprising a charging coil operatively connected to the battery.

The chestpiece 10 further comprises a communication module 42 configured carry out data transfer between the chestpiece 10 and an external device.

The communication module 42 is preferably a wireless communication module 42 configured to utilize a wireless communication protocol to carry out data transfer between the chestpiece 10 and the external device.

In some embodiments, the wireless communication module 42 comprises a short-range wireless communication module configured to utilize wireless short-range communication protocol to carry out the data transfer.

The wireless communication module 42 may be a Bluetooth communication module configured to utilize Bluetooth communication protocol for data transfer.

The wireless communication module 42 may also be a Wi-fi communication module configured to utilize Wi-Fi communication protocol for data transfer.

The first microphone 30 is connected or operatively connected to the processing unit 40. The processing unit 40 is configured to receive the auscultated sound signal from the first microphone 30.

The second microphone 32 is connected or operatively connected to the processing unit 40. The processing unit 40 is configured to receive the ambient sound signal from the second microphone 32.

The communication module 42 is connected or operatively connected to the processing unit 40. The communication module 42 is configured to transfer data from the chestpiece 10 or from the processing unit 40 to the external device. The communication module 42 is further configured to receive data to the chestpiece 10 or to the processing unit 40 from the external device.

The power unit 44 is configured to provide power to the processing unit 40, the communication module 42 and to the first and second microphones 30, 32. The power unit 44 may be connected to the processing unit 40, the communication module 42 and to the first and second microphones 30, 32.

The chestpiece further comprise a circuit board 50. The processing unit 40, communication module 42, power unit 44, and the first and second microphones 30, 32 are provided to or connected to the circuit board 50.

The chestpiece 10 is connected to the external device with the communication module 42 via a communication connection 100, as shown on FIG. 2.

The external device is a mobile user device 104 such as a module phone or a tablet. The external device may also be a computer 106 such as a laptop. The external device may also be an external audio device 102 such as headphones or a speaker.

The chestpiece 10 is directly connected to the external device 102, 104, 106 via the communication module 42 by utilizing the communication protocol. Accordingly, the chestpiece 10 and the communication module 42 thereof is configured to transfer the auscultated sound signal or the auscultated sound data to the external device 102, 104, 106.

Accordingly, the external device 102, 104, 106 also comprises a device communication module configured to receive the auscultated sound signal or the auscultated sound data from the chestpiece 10 via the communication connection 100.

In some embodiments, the external device is an external server comprising a server communication module configured to carry out data transfer with the chestpiece 10. In this embodiment, the mobile user device 104, the external audio device 102 or the computer 106 is arranged in data transfer connection with the external server. The mobile user device 104, the external audio device 102 or the computer 106 is arranged to receive the auscultated sound signal or the auscultated sound data from the external server.

In some embodiment, the external audio device 102 is connected with the mobile user device 104 or to the computer 106, and the mobile user device 104 or the computer is connected with the external audio device 102.

The mobile user device 104 or the computer 106 or the external audio device 102 is configured to emit the auscultated sounds of the auscultated signal or auscultated sound data derived from the auscultated signal with a speaker device provided to the mobile user device 104 or the computer 106 or the external audio device 102. Accordingly, chestpiece 10 enables the auscultated sounds to be listened on site where the patient is or at distant location over the communication connection 100.

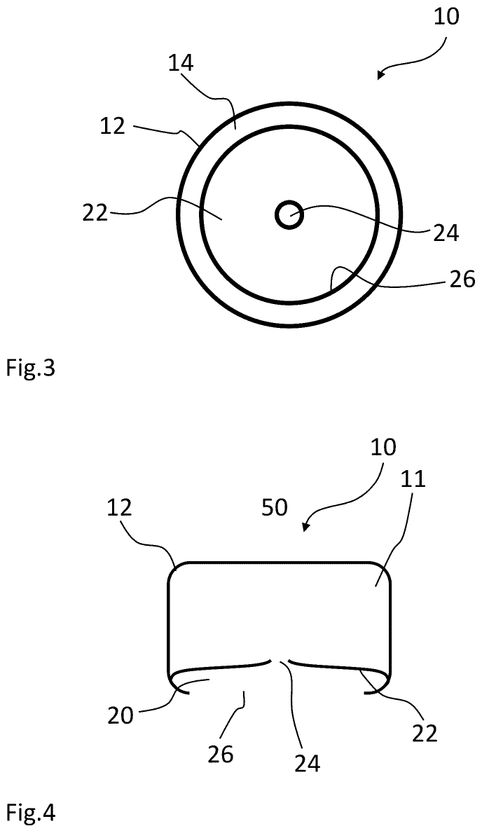

FIG. 3 shows a schematic bottom view of the chestpiece 10. FIG. 4 shows a schematic cross-sectional side view of the chestpiece 10 of FIG. 3. The chestpiece 10 has a circular cylinder shape as shown in FIGS. 1 to 4. IN FIG. 4 the electric components of the chestpiece 10 are not shown.

In the FIG. 3 the diaphragm 15 is not shown. In some embodiments the diaphragm may be omitted and the audio chamber 20 is open to the outer front surface 14 of the chestpiece 10. Thus, the outer front surface 14 comprises a chamber opening 26.

The audio chamber 20 comprises the chamber walls 22 and the top end 24. The top end 24 is provided with the sound opening.

In some embodiments, the bell-shaped audio chamber 20 is without the diaphragm 15.

According to the present invention, the chestpiece 10 is provided with a photoplethysmography (PPG) sensor 60 arranged to detect blood flow volume or change of blood flow volume in the patient body.

The PPG sensor 60 comprises an optical emitter configured to emit light and a light detector arranged to detect light. The optical emitter emits light to a tissue of the patient and the light detector measures the reflected light from the tissue of the patient. The reflected light is proportional to blood volume variations. The PPG sensor generates a photoplethysmography (PPG) signal based on the reflected light measured with the light detector. The PPG signal can be used to diagnose for example cardiac arrhythmias, cardiovascular-related diseases such as atherosclerosis and arterial stiffness, or other cardiac and respiratory irregularities.

In the chestpiece 10 comprises on the outer front surface 14 the PPG sensor. The outer front surface 14 is further provided with the diaphragm 15 arranged to form part of the outer front surface 14 of the chestpiece 10. The PPG sensor 60 is arranged on the outer front surface 14 of the body 12 adjacent to or besides the diaphragm 15.

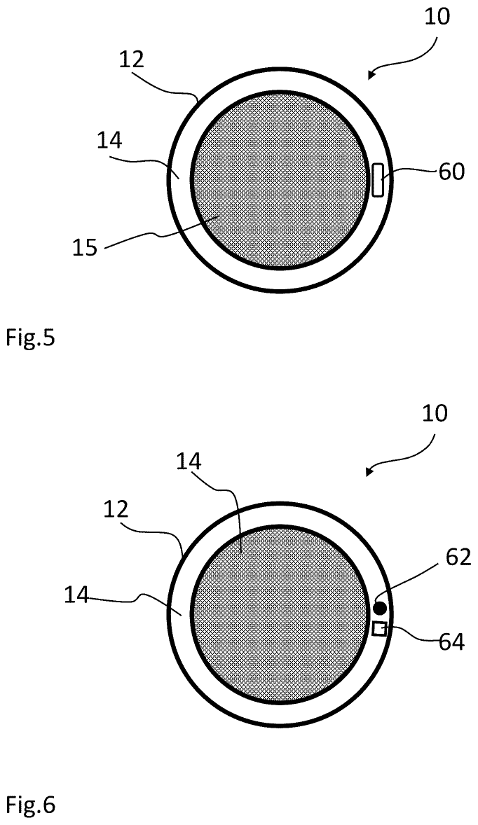

Accordingly in the embodiment of FIG. 5, PPG sensor 60 is arranged to the body 12 on the outer front surface 14.

FIG. 6 shows the embodiment of FIG. 5 in detail. The PPG sensor 60 comprises the optical emitter 62 and the light detector 64. The optical emitter 62 and the light detector 64 are arranged on the outer front surface 14 of the body 12 adjacent to or besides the diaphragm 15. The optical emitter 62 is arranged to emit light in a direction away, or perpendicularly away, from the outer front surface 14 or the diaphragm 15. The light detector 64 is arranged to detect light from a direction towards the outer front surface 14 or the diaphragm 15.

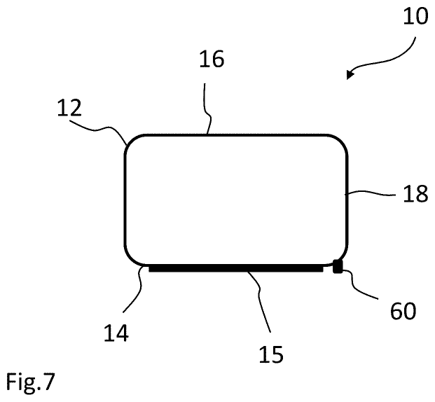

FIG. 7 shows a schematic side view of the embodiment of FIGS. 5 and 6. As shown in FIG. 7, the PPG sensor 60 is arranged on the same level or flush with the diaphragm 15 on the outer front surface 14.

Alternatively, the PPG sensor 60 is arranged to protrude outwards from the diaphragm 15.

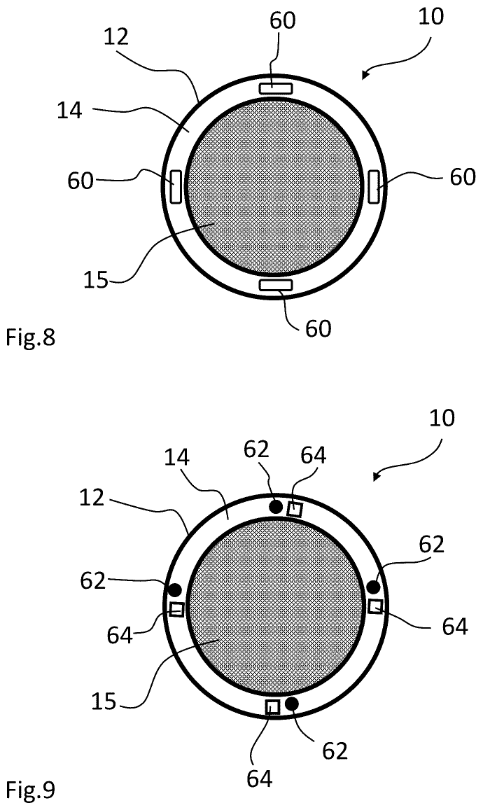

FIGS. 8 and 9 show another embodiment in which the chestpiece 10 comprises four PPG sensors 60. The outer front surface 14 is provided with the diaphragm 15 arranged to form part of the outer front surface 14 of the chestpiece 10. The PPG sensors 60 are arranged on the outer front surface 14 of the body 12 adjacent to or besides the diaphragm 15, similarly as in FIGS. 5 to 7.

FIG. 9 shows the embodiment of FIG. 8 in detail. The PPG sensors 60 comprise the optical emitter 62 and the light detector 64. The optical emitters 62 and the light detectors 64 are arranged on the outer front surface 14 of the body 12 adjacent to or besides the diaphragm 15. The optical emitters 62 are arranged to emit light in a direction away, or perpendicularly away, from the outer front surface 14 or the diaphragm 15. The light detectors 64 are arranged to detect light from a direction towards the outer front surface 14 or the diaphragm 15.

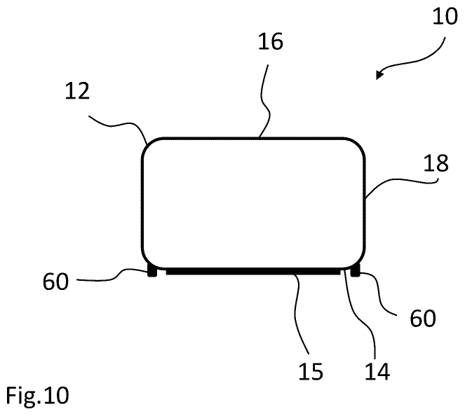

FIG. 10 shows a schematic side view of the embodiment of FIGS. 8 and 9. As shown in FIG. 10, the PPG sensors 60 are arranged on the same level or flush with the diaphragm 15 on the outer front surface 14.

Alternatively, the PPG sensors 60 are arranged to protrude outwards from the diaphragm 15 on the outer front surface 14.

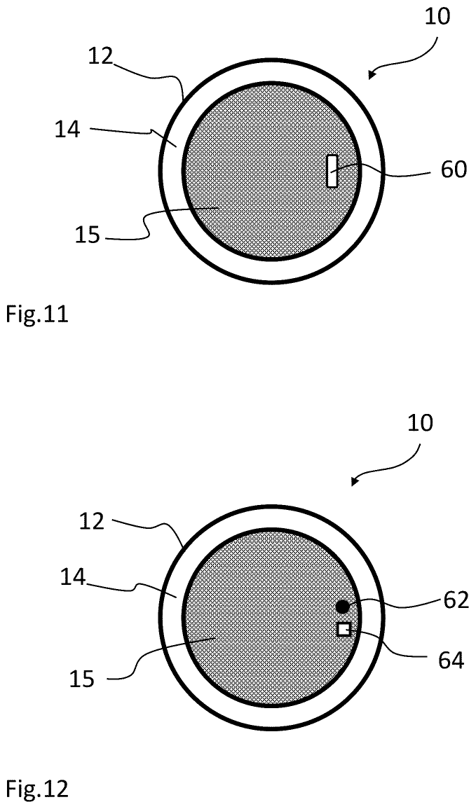

FIG. 11 shows a further embodiment of the present invention. The chestpiece 10 comprises on the outer front surface 14 the PPG sensor. The outer front surface 14 is further provided with the diaphragm 15 arranged to form part of the outer front surface 14 of the chestpiece 10. The PPG sensor 60 is arranged in connection with the diaphragm 15.

The diaphragm 15 comprises an outer diaphragm surface 17 arranged to form at least part of the outer front surface 14 of chestpiece 110, and an inner diaphragm surface 19 towards the audio chamber 20. In the embodiment of FIGS. 11 to 16 the PPG sensor 60 is arranged on the side of the inner diaphragm surface 19 of the diaphragm 15 and arranged to detect blood flow volume in the patient body through the diaphragm 15.

The PPG sensor 60 is arranged to emit light in the direction away, or perpendicularly away, from the outer front surface 14

In the embodiments of FIGS. 11 to 18, the optical emitter 62 is arranged on the side of the inner diaphragm surface 19 of the diaphragm 15 and arranged to emit light through the diaphragm 15. The light detector 64 is arranged is arranged on the side of the inner diaphragm surface 19 of the diaphragm 15 and arranged to detect light through the diaphragm 15.

The optical emitter 62 is arranged to emit light in the direction away, or perpendicularly away, from the outer front surface 14.

Accordingly, in the embodiments of FIGS. 11 to 18 the PPG sensor 60 is arranged in the audio chamber 20.

In the embodiments of FIGS. 11, 12, 15 and 16, the PPG sensor 60 is arranged on the inner diaphragm surface 19 and inside the audio chamber 20. The PPG sensor 60 is arranged to emit light in a direction toward the diaphragm 15 and through the diaphragm 15. The PPG sensor is further arranged to detect light from the direction of the diaphragm 15 and through the diaphragm 15.

The PPG sensor is attached to the inner diaphragm surface 19.

FIGS. 11 and 12 show embodiments, in which the diaphragm is provided light permeable such that light emitted form the PPG sensor 60 or the optical emitter 62 thereof and/or the light detected with the light detector is arranged to pass through the diaphragm 15.

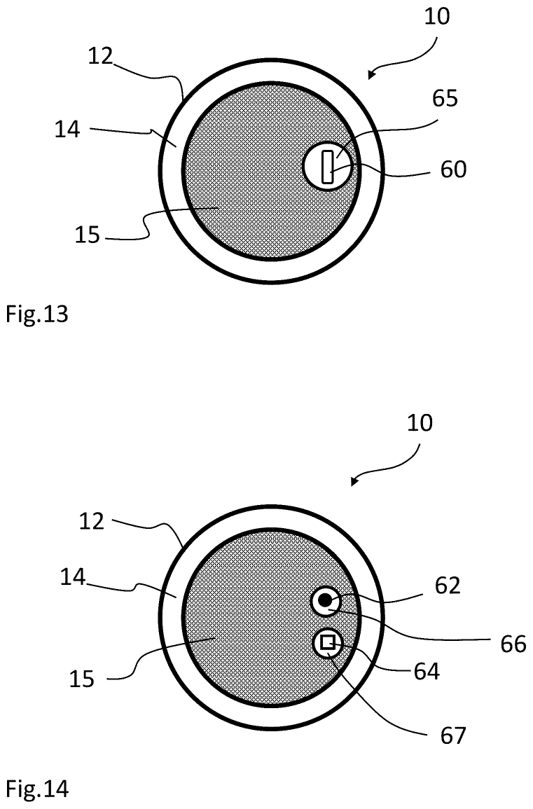

FIGS. 13 and 14 shows different embodiments in which the diaphragm is provided with optical element or elements arranged to enable light travel through the diaphragm.

FIG. 13 shows an embodiment, in which the PPG sensor 60 is arranged on the side of the inner diaphragm surface 19 and inside the audio chamber 20. The diaphragm 15 is provided with an optical element 65 which is configured to be light permeable, transparent or translucent. Thus, light emitted by the PPG sensor 60 can travel through diaphragm 15 via the optical element 15. Similarly, the PPG sensor 60 can detect or measure reflected flight through the diaphragm 15 via the optical element 65.

Accordingly, the PPG sensor 60 is directed towards the optical element 65.

The optical element 65 may be a lens provided to the diaphragm 15.

FIG. 14 shows an alternative embodiment, in which the diaphragm 15 comprises a first optical element 66. The first optical element 66 is a light permeable element. The optical emitter 62 is arranged on the side of the inner diaphragm surface 19 of the diaphragm 15 and arranged to emit light through the first optical element 66. The diaphragm 15 also comprises a second optical element 67. The second optical element 67 is a light permeable element, the light detector 64 is arranged on the side of the inner diaphragm surface 19 of the diaphragm 15 and arranged to detect light through the second optical element 67.

The first and second optical elements 66, 67 may be lenses having different optical characteristics.

The optical emitter 62 is directed towards the first optical element 66 and arranged to emit light through the first optical element 66.

The light detector 64 is directed towards the second optical element 67 and arranged to detect light through the second optical element 67.

The PPG sensor 60 may be attached to the inner surface of the optical element 65.

Alternatively, the PPG sensor 60 may be provided inside the audio chamber at a distance from the optical element 65.

Similarly, the light emitter 62 may be attached to the inner surface of the first optical element 66.

The light detector 64 may be attached to the inner surface of the second optical element 67.

Alternatively, the light emitter may be provided inside the audio chamber at a distance from the first optical element 66 and the light detector may be provided inside the audio chamber at a distance from the second optical element 67.

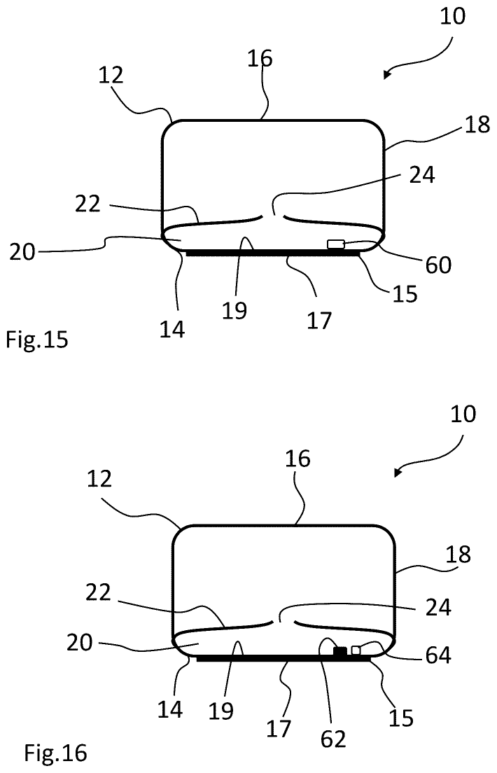

In the embodiments of FIGS. 15 to 22, the diaphragm 15 may be provided light permeable or the diaphragm 15 may be provided the optical element 65, or with the first optical element 66 and/or the second optical element 67.

In the embodiments of FIGS. 11, 12, 15 and 16, the PPG sensor 60 is arranged against or attached to the inner diaphragm surface 19.

In the FIGS. 12 and 16, the optical emitter 62 is arranged on the inner diaphragm surface 19. The optical emitter is arranged to emit light in the direction toward the diaphragm 15 and through the diaphragm 15. The light detector 64 is also arranged on the inner diaphragm surface 19. The light detector 64 is arranged to detect light from the direction of the diaphragm 15 and through the diaphragm 15.

Both the optical emitter 62 and the light detector 64 are arranged against the inner diaphragm surface 19.

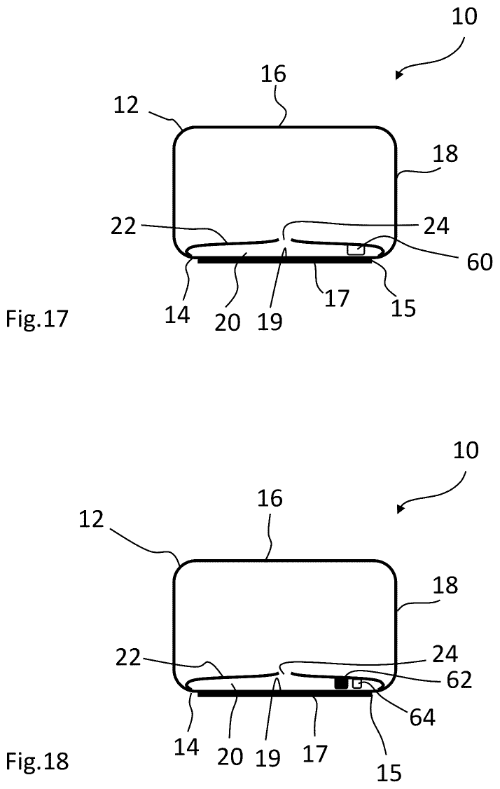

FIGS. 17 and 18 show schematically another embodiment in which the PPG sensor 60 is arranged to the audio chamber wall 22 opposite the diaphragm 15 and in the audio chamber 20. The PPG sensor 60 is arranged to emit light in the direction toward the diaphragm 15 and through the diaphragm 15. The PPG sensor is further arranged to detect light from the direction of the diaphragm 15 and through the diaphragm 15.

The PPG sensor 60 is arranged at a distance from the inner diaphragm surface 19.

The PPG sensor 60 is arranged to emit light in the direction away, or perpendicularly away, from the outer front surface 14 As shown in FIG. 18, the optical emitter 62 is arranged to the audio chamber wall 22 opposite the diaphragm 15. The optical emitter 62 is arranged to emit light in the direction toward the diaphragm 15 and through the diaphragm 15. The light detector 64 is also arranged to the audio chamber wall 22 opposite the diaphragm 15. The light detector 64 is arranged to detect light from the direction of the diaphragm 15 and through the diaphragm 15.

Th light emitter 62 and the light detector are arranged at a distance from the inner diaphragm surface 19.

The optical emitter 62 is arranged to emit light in the direction away, or perpendicularly away, from the outer front surface 14.

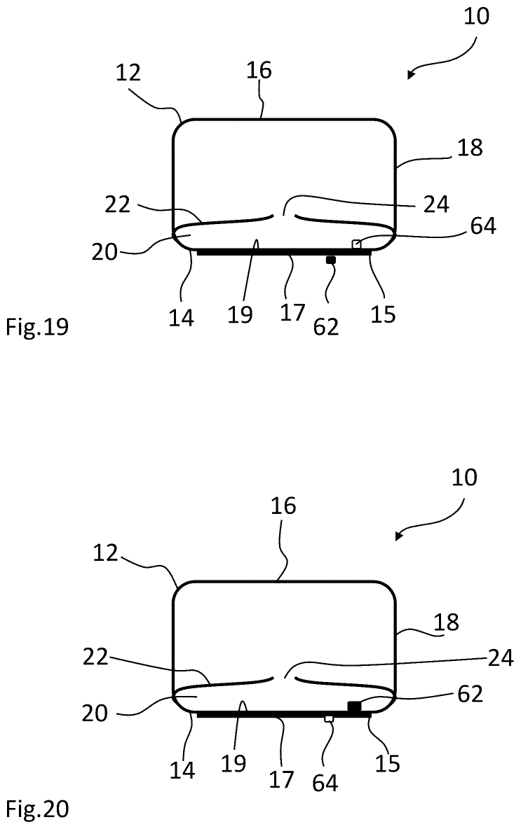

FIGS. 19 to 22 show alternative embodiments of the present invention.

In the embodiment of FIG. 19, the optical emitter 62 of the PPG sensor 60 is arranged on the outer diaphragm surface 17of the diaphragm 15. The optical emitter 62 is arranged to emit light in the direction away, or perpendicularly away, from the outer front surface 14 or the diaphragm 15. The light detector 64 is arranged on the inner diaphragm surface 19. The light detector 64 is arranged to detect light from the direction of the diaphragm 15 and through the diaphragm 15. The light detector 64 are arranged against the inner diaphragm surface 19.

Thus, the light detector 64 is arranged in the audio chamber 20 and the optical emitter 62 is arranged outside the audio chamber 22.

In the embodiment of FIG. 20, the light detector 64 of the PPG sensor 60 is arranged on the on the outer diaphragm surface 17 of the diaphragm 15. The light detector 64 is arranged to detect light from the direction towards, or perpendicularly towards, the outer front surface 14 or the diaphragm 15. The optical emitter 62 is arranged on the inner diaphragm surface 19. The optical emitter 62 is arranged to emit light towards the diaphragm 15 and through the diaphragm 15. The optical emitter 62 are arranged against the inner diaphragm surface 19.

Thus, the optical emitter 62 is arranged in the audio chamber 20 and the light detector 64 is arranged outside the audio chamber.

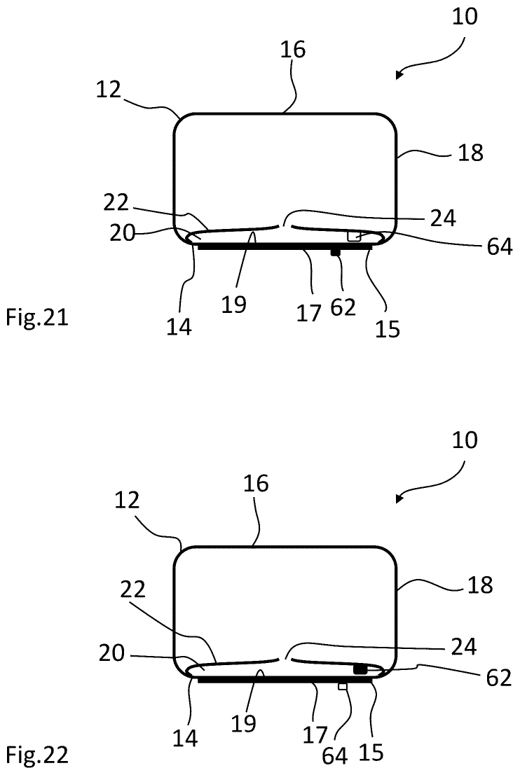

In the embodiment of FIG. 21, the optical emitter 62 of the PPG sensor 60 is arranged on the outer diaphragm surface 17 of the diaphragm 15. The optical emitter 62 is arranged to emit light in the direction away, or perpendicularly away, from the outer front surface 14 or the diaphragm 15. The light detector 64 is arranged on side of the inner diaphragm surface 19 and in the audio chamber 20. The light detector 64 is arranged to the audio chamber wall 22 opposite the diaphragm 15. The light detector 64 is arranged to detect light from the direction of the diaphragm 15 and through the diaphragm 15.

Thus, the light detector 64 is arranged in the audio chamber 20 and the optical emitter 62 is arranged outside the audio chamber 22.

FIG. 22 shows a further embodiment, in which the light detector 64 of the PPG sensor 60 is arranged on the outer diaphragm surface 17 of the diaphragm 15. The light detector 64 is arranged to detect light from the direction towards, or perpendicularly towards, the outer front surface 14 or the diaphragm 15. The optical emitter 62 is arranged on side of the inner diaphragm surface 19. The optical emitter 62 is arranged to the audio chamber wall 22 opposite the diaphragm 15. The optical emitter 62 is arranged to emit light towards the diaphragm 15 and through the diaphragm 15.

Thus, the optical emitter 62 is arranged in the audio chamber 20 and the light detector 64 is arranged outside the audio chamber 22.

It should be noted that in some embodiments, the PPG sensor 60 may be arranged on the outer diaphragm surface 17. Alternatively, the optical emitter 62 may be arranged on the outer diaphragm surface 17, and the light detector 64 on the outer diaphragm surface 17 of the diaphragm 15 or on the side of the inner diaphragm surface 19. Further alternatively, the light detector may be arranged on the outer diaphragm surface 17, and the optical emitter 62 on the outer diaphragm surface 17 of the diaphragm 15 or on the side of the inner diaphragm surface 19.

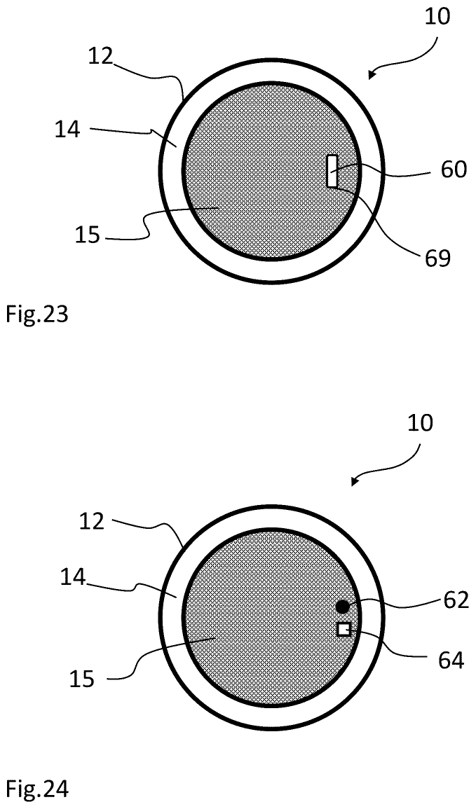

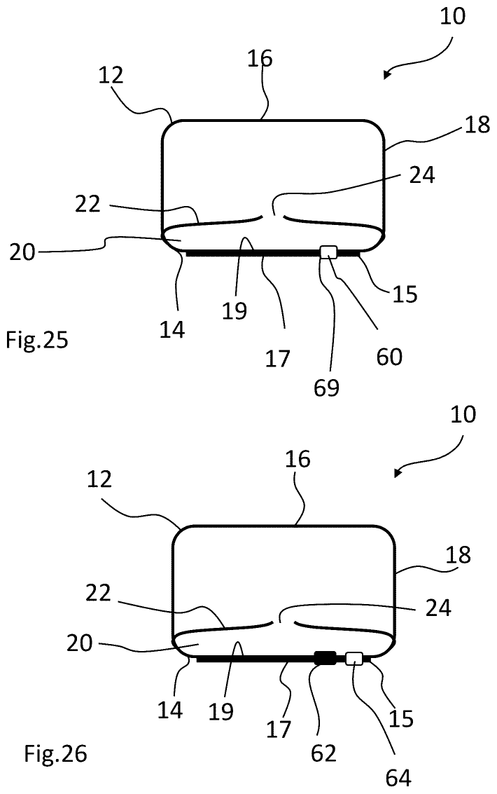

FIGS. 23 to 26 show alternative embodiment. The PPG sensor 60 is arranged to pierce the diaphragm 15. Accordingly, the PPG sensor 60 extends from the inner diaphragm surface 19 to the outer diaphragm surface 17.

The PPG sensor forms part of the diaphragm 15.

Similarly, the light emitter 62 and the light detector 64 are arranged to pierce the diaphragm 15. Accordingly, the light emitter 62 and the light detector 64 extend from the inner diaphragm surface 19 to the outer diaphragm surface 17, as shown in FIGS. 24 and 26.

The light emitter 62 and the light detector 64 form part of the diaphragm 15.

In some embodiments, the diaphragm 16 comprises an opening 69, and the PPG sensor 60, or the light emitter 62 and the light detector 64 are arranged or attached to the opening 69 to pierce the diaphragm 15 via the opening 69.

Thus, the PPG sensor 60, the light emitter 62 and the light detector 64, are provided or attached to the diaphragm 15.

In further embodiments, the PPG sensor 60, the light emitter 62 and the light detector 64, are provided or attached on the outer diaphragm surface 17 of the diaphragm 15.

In the present invention, the PPG sensor 60 is configured to generate the PPG signal based on the detected blood flow volume in the patient body or based on the light detected with the light detector 64 of the PPG sensor 60.

The chestpiece 10 is therefore configured to simultaneously generate the PPG signal with the PG sensor 60 and the auscultated sound signal with the first microphone 30.

The processing unit 40 is configured to receive the PPG signal from the PPG sensor 60.

Accordingly, chestpiece 10 is configured to simultaneously generate the PPG signal with the PPG sensor 60 and the auscultated sound signal with the first microphone 30.

The wireless communication module 42 may be configured to transmit the PPG signal and the auscultated sound signal from the chestpiece 10 to the external receiver simultaneously and/or in real-time.

The processing unit 40 may also be configured to process the PPG signal and the auscultated sound signal into combined output data and the wireless communication module 42 may be configured to transmit the output data to the external receiver.

The invention has been described above with reference to the examples shown in the figures. However, the invention is in no way restricted to the above examples but may vary within the scope of the claims.

1. A chestpiece for an electronic stethoscope, comprising:
a body,
an audio chamber inside the body for detecting auscultated sound from a patient body,
a first microphone provided in connection with the sound chamber for converting the auscultated sound into an auscultated sound signal, and
a processing unit configured to receive the auscultated sound signal from the first microphone,
the body comprises an outer front surface arranged to be placed against the patient body, the audio chamber is provided in connection with the outer front surface for detecting the auscultated sound from the patient body via the outer front surface and the outer front surface comprises a diaphragm arranged to form a front chamber wall of the audio chamber,
wherein the chestpiece comprises a photoplethysmography sensor arranged to detect blood flow volume in the patient body, and that the photoplethysmography sensor is arranged in connection with the diaphragm.

2. The chestpiece according to claim 1, wherein the photoplethysmography sensor comprises an optical emitter configured to emit light and a light detector arranged to detect light.

3. The chestpiece according to claim 2, wherein:
the optical emitter is configured to emit infrared light; or
the optical emitter is configured to emit infrared green light; or
the optical emitter comprises an infrared light emitting diode; or
the optical emitter comprises a green light emitting diode.

4. The chestpiece according to claim 1, wherein the chestpiece comprises two or more photoplethysmography sensors.

5. The chestpiece according to claim 1, wherein: the photoplethysmography sensor is arranged to the diaphragm.

6. The chestpiece according to claim 5, wherein the photoplethysmography sensor is attached to the diaphragm-.

7. The chestpiece according to claim 5, wherein:
the photoplethysmography sensor is arranged to pierce the diaphragm; or
the diaphragm comprises an opening, and the photoplethysmography sensor is arranged to the opening.

8. The chestpiece according to claim 1, wherein the diaphragm comprises an outer diaphragm surface arranged to form at least part of the outer front surface of the body, and an inner diaphragm surface towards the audio chamber, and that:
the photoplethysmography sensor is arranged on the side of the inner diaphragm surface of the diaphragm and arranged to detect blood flow volume in the patient body through the diaphragm; or
the optical emitter is arranged on the side of the inner diaphragm surface of the diaphragm and arranged to emit light through the diaphragm, and the light detector is arranged on the side of the inner diaphragm surface of the diaphragm arranged to detect light through the diaphragm; or
the optical emitter is arranged on the side of the inner diaphragm surface of the diaphragm and arranged to emit light through the diaphragm, and the light detector is arranged on the side of the outer diaphragm surface of the diaphragm or on the outer diaphragm surface of the diaphragm; or
the light detector is arranged on the side of the inner diaphragm surface of the diaphragm and arranged to detect light through the diaphragm and the optical emitter is arranged on the side of the outer diaphragm surface of the diaphragm or on the outer diaphragm surface of the diaphragm.

9. The chestpiece according to claim 8, wherein:
the diaphragm comprises an optical element the optical element being a light permeable element, the photoplethysmography sensor is arranged on the side of the inner diaphragm surface of the diaphragm and configured to emit light through the optical element, and detect light through the optical element; or
the diaphragm comprises a first optical element the first optical element being a light permeable element, the optical emitter is arranged on the side of the inner diaphragm surface of the diaphragm and arranged to emit light through the first optical element; or
the diaphragm comprises a second optical element the second optical element being a light permeable element, the light detector is arranged on the side of the inner diaphragm surface of the diaphragm and arranged to detect light through the second optical element; or
the diaphragm comprises a first optical element the first optical element being a light permeable element, the optical emitter is arranged on the side of the inner diaphragm surface of the diaphragm and arranged to emit light through the first optical element, and
the diaphragm comprises a second optical element the second optical element being a light permeable element, the light detector is arranged on the side of the inner diaphragm surface of the diaphragm and arranged to detect light through the second optical element.

10. The chestpiece according to claim 1, wherein the diaphragm comprises an outer diaphragm surface arranged to form at least part of the outer front surface of the body, and an inner diaphragm surface towards the audio chamber, and that:
the photoplethysmography sensor is arranged on the outer diaphragm surface of the diaphragm; or
the optical emitter is arranged on the outer diaphragm surface of the diaphragm, and the light detector is arranged on the outer diaphragm surface of the diaphragm.

11. The chestpiece according to claim 1, wherein the photoplethysmography sensor is configured to generate a photoplethysmography signal based on the detected blood flow volume in the patient body, and the processing unit is configured to receive the photoplethysmography signal from the photoplethysmography sensor.

12. The chestpiece according to claim 11, wherein chestpiece is configured to simultaneously generate the photoplethysmography signal with the photoplethysmography sensor and the auscultated sound signal with the first microphone.

13. The chestpiece according to claim 11, wherein:
the chestpiece comprises a wireless communication module configured to transmit the photoplethysmography signal and the auscultated sound signal from the chestpiece to an external receiver; or
the processing unit is configured to process the photoplethysmography signal and the auscultated sound signal into output data and the wireless communication module is configured to transmit the output data to an external receiver.

14. The chestpiece according to claim 11, wherein the wireless communication module comprises:
a short-range wireless communication module; or
a Bluetooth communication module; or
a Wi-Fi communication module.

15. An electronic stethoscope, wherein the electronic stethoscope comprises:
a chestpiece comprising:
a body
an audio chamber inside the body for detecting auscultated sound from a patient body,
a first microphone provided in connection with the sound chamber for converting the auscultated sound into an auscultated sound signal,
a photoplethysmography sensor arranged to detect blood flow volume in the patient body and generate a photoplethysmography signal based on the detected blood flow volume,
a processing unit configured to receive the auscultated sound signal from the first microphone and the photoplethysmography signal from the photoplethysmography sensor,
a wireless communication module configured to transmit in wireless manner from the chestpiece the photoplethysmography signal and the auscultated sound signal or output data generated in the processing unit from the photoplethysmography signal and the auscultated sound signal,
the body comprises an outer front surface arranged to be placed against the patient body, the audio chamber is provided in connection with the outer front surface for detecting the auscultated sound from the patient body via the outer front surface,
the outer front surface comprises a diaphragm arranged to form a front chamber wall of the audio chamber,
the photoplethysmography sensor is arranged in connection with the diaphragm, and
the electronic stethoscope further comprises an external user device configured to receive the photoplethysmography signal and the auscultated sound signal or the output data from the chestpiece in wireless manner.

16. The electronic stethoscope according to claim 15, wherein the external user device is:
a mobile user device or a computer configured to receive the photoplethysmography signal and the auscultated sound signal or the output data from the chestpiece in wireless manner; or
an audio output device to receive the photoplethysmography signal and the auscultated sound signal or the output data from the chestpiece in wireless manner.

17. The electronic stethoscope according to claim 16, wherein the external user device is the mobile user device or the computer configured to receive the photoplethysmography signal and the auscultated sound signal or the output data from the chestpiece in wireless manner and that:
the mobile user device or the computer is configured to transmit the photoplethysmography signal and the auscultated sound signal or the output data to the audio output device.

18. The electronic stethoscope according to claim 15, wherein the chestpiece comprises:
a body,
an audio chamber inside the body for detecting auscultated sound from a patient body,
a first microphone provided in connection with the sound chamber for converting the auscultated sound into an auscultated sound signal, and
a processing unit configured to receive the auscultated sound signal from the first microphone,
the body comprises an outer front surface arranged to be placed against the patient body, the audio chamber is provided in connection with the outer front surface for detecting the auscultated sound from the patient body via the outer front surface, and
the outer front surface comprises a diaphragm arranged to form a front chamber wall of the audio chamber,
wherein the chestpiece comprises a photoplethysmography sensor arranged to detect blood flow volume in the patient body, and that the photoplethysmography sensor is arranged in connection with the diaphragm.
